(19) United States
(12) Patent Application Publication
(10) Pub. No.: US 2015/0196489 A1
(43) Pub. Date: Jul. 16, 2015

(54) POLYMER MATRICES FOR CONTROLLING CRYSTALLIZATION

(71) Applicant: Massachusetts Institute of Technology, Cambridge, MA (US)

(72) Inventors: Huseyin Burak Eral, Cambridge, MA (US); Vilmali Lopez-Mejias, Cambridge, MA (US); Allan Stuart Myerson, Boston, MA (US); Bernhardt Levy Trout, Lexington, MA (US); Patrick S. Doyle, Sudbury, MA (US); Marcus O'Mahony, Cambridge, MA (US)

(73) Assignee: Massachusetts Institute of Technology, Cambridge, MA (US)

(21) Appl. No.: 14/575,069

(22) Filed: Dec. 18, 2014

Related U.S. Application Data
(60) Provisional application No. 61/917,554, filed on Dec. 18, 2013.

(51) Int. Cl. A61K 9/14; A61K 31/216

(52) U.S. Cl. CPC A61K 9/146; A61K 31/216

(57) ABSTRACT

Compositions, methods, and systems for controlling crystallization of an agent are generally described. In some embodiments, an agent is crystallized in the presence of polymer matrices, such as polymer particles. The polymer matrix may influence at least a portion of the crystallization process and/or the resulting composition. In some such embodiments, the polymer matrix allows one or more aspect of the process and/or composition to be controlled and/or altered. For instance, the polymer matrix may act as a crystallization promoter and/or acceptable carriers of the crystallized agent. In certain embodiments, the polymer matrix described herein, can be used with any agent regardless of its chemical and/or physical properties.

### RELATED APPLICATIONS

This application claims the benefit of U.S. Provisional Patent Application Ser. No. 61/917,554, filed Dec. 18, 2013, entitled “Polymer Matrices for Controlling Crystallization,” which is incorporated herein by reference in its entirety.

### TECHNICAL FIELD

Compositions, methods, and systems for controlling crystallization of an agent (e.g., pharmaceutically active agent) are generally described.

### BACKGROUND

Crystalline materials are omnipresent in nature, consumer products, and industrial products and practices. Many crystalline materials are formed via a crystallization process. In numerous areas of science and technology, such as the production of pharmaceuticals and other chemicals, the ability to control crystallization is desired. One method for controlling crystallization is to target one or more step in the crystallization process. Most crystallization processes start with heterogeneous nucleation, which occurs at preferential nucleation sites and is a critical step in the crystallization process. However, the process of heterogeneous nucleation, is complex and not well understood.

Accordingly, improved compositions and methods for controlling crystallization are needed.

### SUMMARY

Compositions, methods, and systems for controlling crystallization of an agent are provided. The subject matter of the present invention involves, in some cases, interrelated products, alternative solutions to a particular problem, and/or a plurality of different uses of one or more systems and/or articles.

In one aspect compositions are provided. In some embodiments, the composition comprises a polymer particle comprising crystals of a pharmaceutically active agent, wherein the crystals have an average diameter that is greater than an average mesh size of the polymer particle and wherein the average diameter has a coefficient of variation less than or equal to about 10%.

In certain embodiments, the composition comprises crystals of a pharmaceutically active agent dispersed throughout a cross-linked polymer matrix, wherein the solubility of a polymer matrix precursor in a solvent prior to crosslinking is at least 2 times greater than the solubility of the pharmaceutically active agent in the solvent.

In another aspect, a pharmaceutical composition is provided. In some embodiments, the pharmaceutical composition comprises a particulate polymer carrier and a pharmaceutically active agent primarily encapsulated by the particulate polymer carrier, wherein the active agent has been crystallized in the presence of the particulate polymer carrier.

In another aspect crystallization methods are provided. In some embodiments, the method comprises crystallizing a pharmaceutically active agent in a fluid droplet within a polymer particle.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic illustration of a method of crystallizing an agent, according to some embodiments;

FIG. 1B is a schematic illustration of a method of crystallizing an agent, according to some embodiments;

FIG. 2A is a schematic illustration of a method of crystallizing an agent, according to certain embodiments;

FIG. 2B is a schematic illustration of a method of crystallizing an agent, according to certain embodiments;

FIG. 3 is an illustration of methods of associating an agent with a polymer matrix, according to some embodiments;

FIG. 4A is a graph of rheological measurements, according to one set of embodiment;

FIG. 4B is a graph of the average polymer matrix mesh size versus concentration, according to one set of embodiment;

FIG. 5 is a graph of nucleation induction probability for various polymer matrix formulations, according to certain embodiments;

FIG. 6A is a graph of agent loading for various polymer matrix formulations, according to one set of embodiments;

FIG. 6B is a graph of agent loading for various polymer matrix formulations, according to one set of embodiments;

FIG. 7A is a schematic of an emulsion, according to certain embodiments;

FIG. 7B is an image of an emulsion, according to certain embodiments;

FIG. 7C is an image of polymer matrix, according to certain embodiments;

FIG. 8A is a graph of agent loading, according to one set of embodiments;

FIG. 8B is a graph of agent loading, according to one set of embodiments;

FIG. 9A is a schematic of a crystallization method, according to certain embodiments;

FIG. 9B are images of a polymer matrix at various stages, according to certain embodiments;

FIG. 10A is a graph of X-ray diffraction data, according to one set of embodiments;

FIG. 10B is a graph of differential scanning calorimetry data, according to one set of embodiments;

FIG. 10C is a graph of a mean crystal size for various droplet diameters, according to one set of embodiments;

FIG. 10D is a graph of a droplet diameter at various agent concentrations, according to one set of embodiments;

FIG. 11A is a graph of crystal dissolution, according to certain embodiments;

FIG. 11B is a graph of crystal dissolution, according to certain embodiments; and

FIG. 12 is a graph of crystal dissolution, according to certain embodiments.

### DETAILED DESCRIPTION

Compositions, methods, and systems for controlling crystallization of an agent (e.g., pharmaceutically active agent) are generally described. In some embodiments, an agent is crystallized in the presence of polymer matrices, such as polymer particles. The polymer matrix may influence at least a portion of the crystallization process (e.g., nucleation or crystal growth) and/or the resulting composition (e.g., crystals). In some such embodiments, the polymer matrix allows one or more aspect of the process and/or composition to be controlled and/or altered. For instance, the polymer matrix may act as a crystallization promoter (e.g., heteronucleant) and/or acceptable carriers of the crystallized agent. In certain embodiments, the polymer matrix described herein, can be used with any agent regardless of its chemical and/or physical properties (e.g., solubility). Methods utilizing the polymer matrices may allow certain aspects of the resulting composition (e.g., crystal size, weight percentage of crystals) to be altered and/or controlled.

In many applications, it is advantageous to control and/or alter the crystallization process and/or the resulting composition. Though most crystallization processes are heterogeneous and might be altered through the use of appropriately designed heteronucleant, the crystallization process, including nucleation behavior, is mostly unpredictable. The unpredictability of crystallization as well as the practical constraints on crystallization methods (e.g., in industrial practice) hamper the rational design of suitable materials capable of influencing crystallization. Therefore, conventional crystallization methods seek to control crystallization by adjusting parameters such as saturation level, temperature profile, solvent selection, stirring speed, etc. In general, the determination of appropriate crystallization parameters can be time-consuming and/or the parameters may have to be determined for each agent or class of agents.

It has been discovered, within the context of the present invention, that certain polymer matrices can control and/or alter the crystallization process (e.g., nucleation kinetics) and/or the resulting crystals (e.g., crystal size) without having to design a new polymer matrix for each agent or class of agents. In some embodiments, the polymer matrix is compatible with the intended use of the crystallized agent, such that at least a portion of the polymer matrix is not removed prior to use of the crystallized agent. In such cases, the need for post-crystallization processing relating to the polymer matrix is reduced or eliminated.

As described herein, an agent may be crystallized in the presence of polymer matrices (e.g., hydrogel particles). In some embodiments, a method of crystallizing an agent involves associating the agent with a polymer matrix prior to crystallization and inducing crystallization of the agent while it is associated with the polymer matrix. For example, the agent may be at least partially encapsulated by a polymer matrix (e.g., polymer particle) prior to and during crystallization. In some embodiments, after crystallization, the polymer matrix may comprise crystals of the agent. In some instances, the diameter of at least a portion of the crystals (e.g., average crystal diameter) may be greater than the pore size (i.e., mesh size) of the polymer matrix. In some such embodiments, at least a portion of the crystals are confined within and/or primarily encapsulated by the polymer matrix (e.g., polymer particle).

As noted above, the agent may be associated with the polymer matrices prior to crystallization. In some embodiments, the agent and the polymer matrices may be dissolved in a common solvent and allowed to associate. In other embodiments, the solubility of an agent, in at least one solvent, is substantially different than the solubility of at least one precursor of the polymer matrix (e.g., monomers, polymer molecules) that affects the ability of the polymer matrix to be carried in the solvent without precipitating out. For instance, the agent may have a relatively low solubility (e.g., solubility of less than about 1 mg/ml) in a solvent (e.g., aqueous based solvent) and at least one precursor of the polymer matrix may have a relatively high solubility (e.g., greater than about 10 mg/ml) in the solvent (e.g. aqueous based solvent), such that the polymer matrix also has a relatively high solubility in the solvent (e.g., aqueous based solvent). In some such embodiments, an association between the agent and the polymer matrix, at adequate concentrations of the agent and matrix, cannot be readily formed through dissolution in a common solvent.

As used herein, solubility, and accordingly dissolution, with respect to the polymer matrix may refer to the ability of the of the polymer matrix to be carried in the solvent without precipitating out. The solubility may be expressed in terms of concentration of the saturated solution of the polymer matrix at standard conditions.

In some embodiments, an emulsion system may be used to associate a polymer matrix with an agent that has a substantially different solubility in at least one solvent than the polymer matrix or precursor. The agent may have a relatively low solubility in a first solvent in which the polymer matrix or precursor is dissolved. In some such embodiments, the agent is dissolved in a second solvent that is substantially immiscible with the first solvent. The polymer matrix or precursor in the first solvent may be combined with the agent in the second solvent to form an emulsion. In certain embodiments, the dispersed phase of the emulsion is the second solvent comprising the dissolved agent and the continuous phase is the first solvent comprising the polymer matrix or precursor. In other embodiments, the dispersed phase of the emulsion is the first solvent comprising the polymer matrix or precursor and the continuous phase is the second solvent comprising the dissolved agent. In some embodiments, the polymer matrix or precursor may be cross-linked or further cross-linked while in the emulsion (e.g., as the continuous phase). In some embodiments in which the continuous phase comprises the polymer matrix, at least a portion of the dispersed phase comprising the agent may be confined within and/or primarily encapsulated by the polymer matrix.

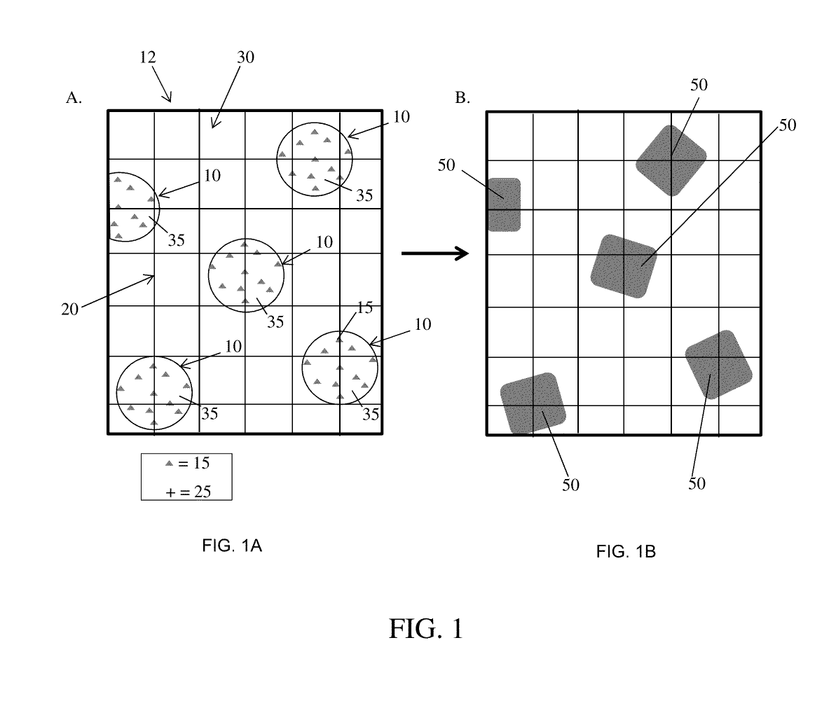

A non-limiting example of a method of crystallizing an agent that has a substantially different solubility than the polymer matrix in at least one solvent is shown in FIG. 1. FIG. 1 is a schematic illustration showing a cross-section of a portion of a single polymer matrix before and after crystallization, according to certain embodiments. As illustrated in FIG. 1, prior to crystallization, an emulsion is formed to associate the polymer matrix with the agent. Fluid droplets 10 comprising an agent 15 (e.g., pharmaceutically active agent) may be dispersed within a continuous phase 12 comprising the polymer matrix 20 having cross-links 25 and a first solvent 30 that is substantially immiscible with second solvent 35 used to form the fluid droplets. In some embodiments, the first solvent is a polar solvent, such as water. In other embodiments, the first solvent is an apolar and/or organic solvent. For example, when the first solvent is a polar solvent (e.g., water), the fluid in the droplet (i.e., second solvent) is an apolar solvent. In another example, when the first solvent is an apolar solvent, the second solvent is a polar solvent. In some embodiments, the mesh size of the polymer matrix relative to the droplet size may cause at least a portion of the fluid droplets to be confined and retained in the polymer matrix.

After the polymer matrix is associated with the agent, the agent in the fluid droplet may be crystallized within the polymer matrix (e.g. polymer particle) by any suitable method known to those of ordinary skill in the art to induce crystallization (e.g., evaporation, temperature shock, chemical interference). Without being bound by theory, it is believed that the fluid droplets within the polymer matrix serve as compartmentalized units where crystallization can be achieved. It is believed that since these compartmentalized units are accessible and retained within the polymer matrix, crystallization can be induced.

In some embodiments, as illustrated in FIG. 1B, a single crystal 50 is formed in the fluid droplet. For instance, a polymer particle comprising twenty fluid droplets has no more than twenty crystals after crystallization. In some embodiments, the resulting crystals are retained within or associated with the polymer matrix. In some such cases, the average diameter of the crystals may be greater than the average mesh size of the polymer matrix. In certain embodiments, the geometry of the crystals may be controlled, in part, by the geometry of fluid droplets. For instance, the geometry of the fluid droplets may influence crystal diameter. Without wishing to be bound by theory, it is believed that the diameter of the fluid droplet sets an upper limit for the crystal diameter and the coefficient of variation in the crystal diameter. That is, the maximum average diameter and coefficient of variation is substantially the same as that of the fluid droplets. It is also believed that the diameter of the crystal is also a function of the concentration of the agent in the dispersed phase, such that the crystal diameter decreases as the concentration of the agent decreases and the crystal size is substantially the same as the droplet size when the fluid in the droplet is saturated with the agent.

In some embodiments, the ratio of the crystal diameter to the diameter of the fluid droplet is less than or equal to about 1:1, less than or equal to about 0.75:1, less than or equal to about 0.5:1, less than or equal to about 0.25:1, less than or equal to about 0.10:1, or less than or equal to about 0.05:1.

In some embodiments, the average diameter of the crystals formed using an emulsion system is less than or equal to about 100 microns, less than or equal to about 10 microns, less than or equal to about 1 micron, less than or equal to about 0.8 microns, less than or equal to about 0.6 microns, less than or equal to about 0.4 microns, less than or equal to about 0.2 microns, less than or equal to about 0.1 microns, less than or equal to about 0.08 microns less than or equal to about 0.05 microns, or less than or equal to about 0.02 microns. In some instances, the average diameter of the crystals formed using an emulsion system may be between about 0.01 microns and about 100 microns, between about 0.01 microns and about 10 microns, between about 0.01 microns and about 1 microns, or between about 0.01 microns and about 0.4 microns.

In some embodiments, the coefficient of variation in the average crystal diameter is less than or equal to about 20%, less than or equal to about 15%, less than or equal to about 10% or less than or equal to about 5%.

As shown in FIG. 1, an emulsion may be used to associate agents and polymer matrices with a substantially different solubility in at least one solvent (e.g., water). Without being bound by theory, it is believed that solubility of the agent and the polymer matrices in the emulsion solvents is an important factor in the formation of a suitable emulsion system. It is believed that the agent should be relatively insoluble in the first solvent (e.g., continuous phase) and highly soluble in the second solvent (e.g., dispersed phase). Conversely, it is believed that the polymer matrix should be relatively insoluble in the second solvent and highly soluble in the first solvent (e.g., continuous phase). For instance, the solubility of the agent in the first solvent and the solubility of the polymer matrix and/or precursor in the second solvent is less than about 0.25 mg/ml, less than about 0.1 mg/ml, less than about 0.05 mg/ml, less than about 0.01 mg/ml, less than about 0.005 mg/ml, or less than about 0.001 mg/ml.

The solubility of the agent in the second solvent and the solubility of the polymer matrix and/or precursor in the first solvent may be greater than or equal to about 0.01 g/ml, greater than or equal to about 0.05 g/ml, greater than or equal to about 0.1 g/ml, greater than or equal to about 0.5 g/ml, greater than or equal to about 1.0 g/ml, greater than or equal to about 5 g/ml, greater than or equal to about 10 g/ml, greater than or equal to about 25 g/ml, greater than or equal to about 50 g/ml, or greater than or equal to about 75 g/ml. In some embodiments, the solubility of the agent in the second solvent and the solubility of the polymer matrix and/or precursor in the first solvent may be between about 0.01 g/ml and about 100 g/ml, between about 0.1 g/ml and about 100 g/ml, between about 1.0 g/mL and about 100 g/mL, or between about 10 g/ml and about 100 g/ml.

In some embodiments, the solubility of a precursor of the polymer matrix and/or the polymer matrix may be at least 2 times, at least 5 times, at least 10 times, at least 25 times, at least 50 times, at least 75 times, at least 100 times, at least 150 times, at least 200 times, at least 250 times, at least 300 times, at least 350 times, at least 400 times, at least 450 times, or at least 500 times greater than the solubility of the agent in the solvent. For example, when the first solvent is water, the solubility of a hydrophilic precursor of the polymer matrix (e.g., polymer molecule) is at least 100 times (e.g. at least 200 times, at least 300 times, at least 400 times, at least 500 times) greater than a hydrophobic agent (e.g., pharmaceutically active agent).

In general, any suitable solvents may be used as a first and a second solvent provided that the first and second solvents are substantially immiscible and the agent and the polymer matrix has the requisite solubility in each solvent. For instance, in some embodiments, the solvent may be selected from FDA approved solvents (e.g., FDA generally regarded as safe (GRAS) solvents, FDA approved class III solvents). Non-limiting examples of suitable polar solvents include water, aqueous based solvents, acetic acid, acetone, dimethylformamide, acetonitrile, ethyl formate, formic acid, dimethyl sulfoxide, dichloromethane, butanol, 3-methyl-2-butanol, ethanol, methanol, pentanol, acetic acid, isopropanol, propanol, 2-methyl-1-propanol, nitromethane, and/or combinations thereof. Non-limiting examples of suitable apolar solvents include ethyl acetate, isobutyl acetate, methyl acetate, propyl acetate, methyl pentane, methylethylketone, methylisobutylketone, isobutyl ketone, cumene, tert-butyl-methylester ether, heptane, hexane, anisole, toluene, chloroform, diethyl ether, benzene, isopropyl acetate, cyclohexane, ether (e.g., ethyl ether), dioxane, tetrahydrofuran, FDA GRAS oils (e.g., corn oil, olive oil) and/or combinations thereof. Those of ordinary skill in the art can select suitable substantially immiscible fluids, using contact angle measurements or the like, based on the description herein.

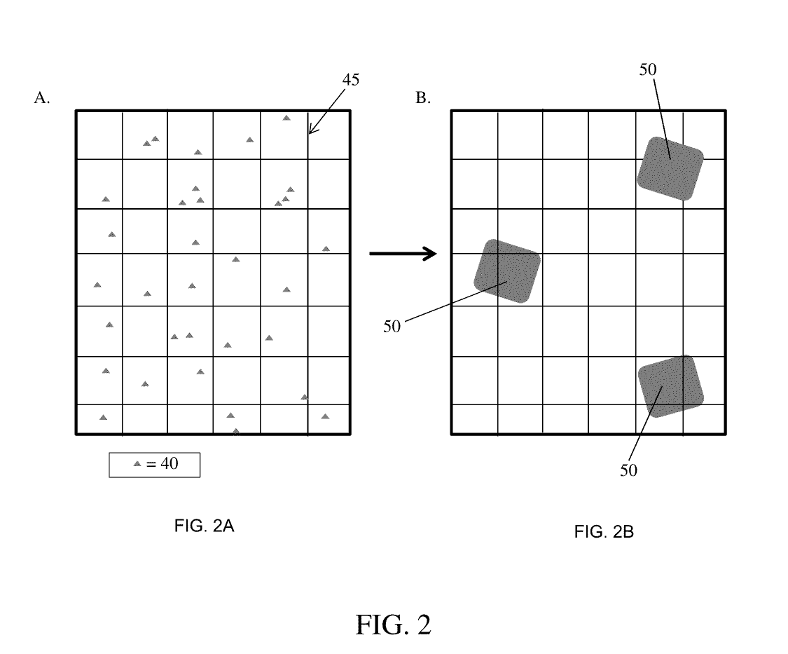

As described herein, the agent and the polymer matrix may have similar solubility in at least one solvent (e.g., water), such that the agent and the polymer matrices can be readily associated at adequate concentrations. In general, any suitable method known to those of skill in the art may be used to cause the agent and polymer matrices to associate. In some instances, the agent and the polymer matrices are incubated in a common solvent and allowed to form selective interactions. In some cases, a precursor of the polymer matrix and the agent are incubated in the common solvent and allowed to form selective interactions. The polymer matrix precursor may be cross-linked after association with the agent to form a plurality of polymer matrices. An exemplary method of crystallizing an agent and polymer matrix in a common solvent is illustrated in FIG. 2. FIG. 2 is a schematic illustration showing a cross-section of a portion of the polymer matrix before and after polymerization. As illustrated in FIG. 2A, prior to crystallization, the agent 40 may be associated with the polymer matrix 45 via selective interaction(s). The agent may be crystallized in the presence of the polymer matrix. As illustrated in FIG. 2B, the resulting crystals 50 may be associated with the polymer matrix. In some embodiments, the crystals may be primarily encapsulated or confined within the matrix.

In some embodiments, the average diameter of the crystals formed using a common solvent is less than or equal to about 600 microns, less than or equal to about 500 microns, less than or equal to about 400 microns, less than or equal to about 300 microns, less than or equal to about 200 microns, less than or equal to about 100 microns, less than or equal to about 50 microns, less than or equal to about 20 microns, less than or equal to about 10 microns, less than or equal to about 1 micron, less than or equal to about 0.8 microns, less than or equal to about 0.6 microns, less than or equal to about 0.4 microns, less than or equal to about 0.2 microns, less than or equal to about 0.1 microns, less than or equal to about 0.05 microns, or less than or equal to about 0.02 microns. In some embodiments, the average diameter of the crystals formed using a common solvent is between about 0.01 microns and about 600 microns, between about 0.02 microns and about 600 microns, between about 0.05 microns and about 600 microns, between about 0.1 microns and about 600 microns, between about 0.01 microns and about 100 microns, between about 0.01 microns and about 10 microns, or between about 0.01 microns and about 1 micron.

In some embodiments, the coefficient of variation in the average crystal diameter is less than or equal to about 20%, less than or equal to about 15%, less than or equal to about 10% or less than or equal to about 5%.

As described herein, the polymer matrices may serve as a crystallization promoter. Without being bound by theory, it is believed that the average nucleation induction time of agent is influenced by the mesh size of the polymer matrix. It is believed that there is an optimal range in mesh size that allows sufficient favorable interaction between the polymer matrix and the agent molecules to occur. It is also believed that optimal range in mesh size allows for agent molecules associated with the polymer molecules in the polymer matrix to come within sufficient proximity to form a nucleus of agent molecules and polymer molecules. For relatively small mesh sizes, an agent molecule may “see” more polymer chains than solvent molecules, which enhances the interaction between the agent molecule and the polymer molecules in the polymer matrix. For relatively large mesh sizes, it is believed that the agent molecules and the polymer molecules in the matrix are separated from each other, such that the interaction between the agent molecules and the polymer molecules is not enhanced.

In some embodiments, crystallizing an agent in the presence of a polymer matrix decreases the average nucleation induction time compared to crystallizing an agent in the absence of the polymer matrix under identical crystallization conditions. For instance, in some embodiments, the percent decrease in the average nucleation induction time may be greater than or equal to about 10%, greater than or equal to about 20%, greater than or equal to about 40%, greater than or equal to about 60%, greater than or equal to about 80%, greater than or equal to about 100%, or greater than or equal to about 150%. In some instances, the percent decrease may be less than or equal to about 200%, less than or equal to about 150%, less than or equal to about 100%, less than or equal to about 80%, less than or equal to about 60%, less than or equal to about 40%, less than or equal to about 20%, less than or equal to about 10%, or less than or equal to about 5%. All combinations of the above-referenced ranges are also possible (e.g., greater than or equal to about 10% and less than or equal to about 200%, greater than or equal to about 10% and less than or equal to about 100%). Crystal nucleation was determined by continuous monitoring of the sample using an inverted microscope. The onset of crystallization was the point at which the first crystal appeared. Statistical analysis methods known to those of ordinary skill in the art were used to calculate the average induction time.

Without being bound by theory, it is believed that the average nucleation induction time of agent is influenced by the mesh size (i.e., pore size) of the polymer matrix. In some embodiments, the mesh size of the polymer matrix may be less than or equal to about 20 nm, less than or equal to about 15 nm, less than or equal to about 12 nm, less than or equal to about 10 nm, or less than or equal to about 8 nm. In some embodiments, the mesh size of the polymer is less than or equal to about 10 nm. Mesh size may be determined via oscillatory rheology using frequency sweep measurements at a fixed strain modeled in terms of the generalized Maxwell model.

In some embodiments, the average mesh size may be less than the average diameter of the fluid droplets in the dispersed phase of the emulsion and/or the crystal size of the crystalized agent. For instance, in some embodiments, the ratio of the average mesh size to the average diameter of the fluid droplets and/or the crystals is less than or equal to about 0.95:1, less than or equal to about 0.8:1, less than or equal to about 0.6:1, less than or equal to about 0.4:1, less than or equal to about 0.2:1, or less than or equal to about 0.1:1.

In some embodiments, the average diameter of the fluid droplets is less than or equal to about 100 microns, less than or equal to about 10 microns, less than or equal to about 1 micron, less than or equal to about 0.8 microns, less than or equal to about 0.6 microns, less than or equal to about 0.4 microns, less than or equal to about 0.2 microns, less than or equal to about 0.1 microns, less than or equal to about 0.05 microns, less than or equal to about 0.02 microns, or less than or equal to about 0.01 microns. In some instances, the average diameter of the fluid droplets is between about 0.01 microns and about 100 microns, between about 0.01 microns and about 10 microns, between about 0.01 microns and about 1 micron, or less than or between about 0.01 microns and about 0.4 microns.

In some embodiments, the coefficient of variation in the average droplet diameter is less than or equal to about 20%, less than or equal to about 15%, less than or equal to about 10% or less than or equal to about 5%.

Methods and systems described herein may allow one or more property of the resulting composition to be controlled. For example, crystallizing an agent in the presence of polymer matrices may allow the crystals to be associated with the polymer matrices after crystallization. In certain embodiments, the majority of the crystals associated with a polymer matrix may be encapsulated within the polymer matrix. For instance, in some embodiments, the percentage of crystals that are encapsulated by the polymer matrix is greater than or equal to about 60%, greater than or equal to about 70%, greater than or equal to about 80%, greater than or equal to about 90%, greater than or equal to about 95%, greater than or equal to about 98%, or greater than or equal to about 99%. The percentage of crystals that are encapsulated by the polymer matrix may be determined using transmission electron microscopy.

In some embodiments, the polymer matrix may serve as carriers for the crystals. For instance, a pharmaceutically active agent may be crystallized in the presence of a polymer particles, such that the pharmaceutically active agent is primarily encapsulated (e.g., greater than or equal to about 80%, greater than or equal to about 90%, greater than or equal to about 95%, greater than or equal to about 98%, or greater than or equal to about 99% encapsulated) in the polymer particles. The polymer particles may serve as particulate carriers for the pharmaceutically active agent.

In some embodiments, association via emulsion may allow the weight percentage of the agent and the polymer matrix in the composition to be controlled. For instance, the weight percentage of the agent may be controlled by varying the concentration of the dispersed phase in the emulsion and/or the concentration of the agent in the dispersed phase. In some embodiments, the emulsion may be formulated such that the resulting composition comprising crystals and polymer matrices has a relatively high weight percentage of crystals (e.g., greater than equal to about 75%). In general, the emulsion may be formulated to produce a composition with any suitable weight percentage of the crystallized agent. For instance, in some embodiments, the weight percentage of crystallized agent is greater than or equal to about 10%, greater than or equal to about 20%, greater than or equal to about 30%, greater than or equal to about 40%, greater than or equal to about 50%, greater than or equal to about 60%, greater than or equal to about 70%, or greater than or equal to about 80%. In some instances, the weight percentage of the crystallized agent is less than or equal to about 90%, less than or equal to about 80%, less than or equal to about 70%, less than or equal to about 60%, less than or equal to about 50%, less than or equal to about 40%, less than or equal to about 30%, or less than or equal to about 20%. All combinations of the above-referenced ranges are also possible (e.g., greater than or equal to about 20% and less than or equal to about 90%). Weight percentage, as used herein, refers to the dry weight percentage.

In some embodiments, association via non-emulsion methods, such as dissolution in a common solvent, may allow the weight percentage of the agent and the polymer matrix in the composition to be controlled. In general, the composition comprising the polymer matrix and the crystallized agent may have any suitable weight percentage of the crystallized agent. For instance, in some embodiments, the weight percentage of crystallized agent is greater than or equal to about 5%, greater than or equal to about 10%, greater than or equal to about 15%, greater than or equal to about 20%, greater than or equal to about 30%, or greater than or equal to about 40%. In some instances, the weight percentage of the crystallized agent is less than or equal to about 50%, less than or equal to about 40%, less than or equal to about 30%, or less than or equal to about 20%. All combinations of the above-referenced ranges are also possible (e.g., greater than or equal to about 5% and less than or equal to about 30%.

In some embodiments, the polymer matrix may be designed to function as a suitable carrier for diverse application (e.g., methods involving the crystallized agent, products containing the crystallized agent). For instance, the polymer matrix may be designed to be, e.g., biocompatible so that it can be used in pharmaceutical compositions and/or consumer products. In some embodiments, the polymer matrix may comprise polymer molecules associated via chemical (e.g., covalent, non-covalent), physical (e.g., entanglement), and/or biological (e.g., receptor-ligand) interactions. In some embodiments, at least a portion of the interactions may form cross-links. In general, the polymer molecules may be cross-linked via any suitable interaction.

In some embodiments, the polymer molecules may be associated via a chemical interaction, such as a chemical bond. The chemical bond may be a covalent bond or non-covalent bond. In some cases, the chemical bond is a non-covalent bond such as a hydrogen bond, ionic bond, dative bond, and/or a Van der Waals interaction. One or more of the polymer molecules may comprise functional groups capable of forming such bonds. It should be understood that covalent and non-covalent bonds between components may be formed by any type of reactions, as known to those of ordinary skill in the art, using the appropriate functional groups to undergo such reactions. Chemical interactions suitable for use with various embodiments described herein can be selected readily by those of ordinary skill in the art, based upon the description herein.

In some embodiments, the polymer molecules may be associated via physical interactions. For example, in some embodiments, at least a portion of the polymer molecules are physically entangled.

In some embodiments, the polymer molecules may be associated via biological interactions, such as a biological binding event (i.e., between complementary pairs of biological molecules). One or more of the polymer molecules may comprise biological molecules capable of forming such bonds. Examples of biological molecules that may form biological bonds between pairs of biological molecules include, but are not limited to, proteins, nucleic acids, glycoproteins, carbohydrates, hormones, and the like. Non-limiting examples include, but are not limited to, a protein/substrate pair, a nucleic acid/nucleic acid pair, a protein/nucleic acid pair, a peptide/peptide pair, a protein/protein pair, a small molecule/protein pair, a receptor/hormone pair, a biotin/avidin pair, a biotin/streptavidin pair, a drug/target pair, small molecule/peptide pair, a small molecule/protein pair, and/or combinations thereof. Biological interactions between polymer molecules for use in the embodiments described herein can be selected readily, by those of ordinary skill in the art, based upon the description herein as their function, examples of such biological interactions, and knowledge herein and in the art as to simple techniques for identifying suitable biological interactions.

It should be understood that the polymer matrix may be formed from more than one type of polymer molecule and may have any suitable shape (e.g., particle, planar, non-planar) or dimension. For example, in some embodiments, the polymer matrices may be in particulate form. In some such embodiments, the particles may have an average diameter of greater than or equal to about 0.1 microns, greater than or equal to about 1 micron, greater than or equal to about 10 micron, greater than or equal to about 50 micron, greater than or equal to about 100 micron, greater than or equal to about 200 micron, greater than or equal to about 400 micron, greater than or equal to about 600 micron, greater than or equal to about 800 micron, or greater than or equal to about 1000 micron. In some instances, the average diameter of the polymer particles is less than or equal to about 3,000 microns, less than or equal to about 2,000 microns, less than or equal to about 1,000 microns, less than or equal to about 800 microns, less than or equal to about 600 microns, less than or equal to about 400 microns, less than or equal to about 200 microns, less than or equal to about 100 microns, less than or equal to about 50 microns, less than or equal to about 10 microns, or less than or equal to about 1 micron. All combinations of the above-referenced ranges are also possible (e.g., greater than or equal to about 1 micron and less than or equal to about 1,000 microns, greater than or equal to about 1 micron and less than or equal to about 600 microns, greater than or equal to about 0.1 microns and less than or equal to about 600 microns).

In some embodiments, at least a portion of the crystal encapsulated within the polymer matrix has a portion that protrudes outside of the polymer matrix. In some such embodiments, a polymer shell can be formed around the polymer matrix to cover and mechanically protect the exposed portions of the crystals. In some embodiments, the polymer shell may be formed from the same or different polymer molecules as the polymer matrix. In some embodiments, the polymer shell may also comprise an agent. In some instances, the agent in the shell may be any agent described herein with respect to the polymer matrices. In other embodiments, the polymer shell does not comprise an agent. In some instances, the polymer shell may be cross-linked. In other instances, the polymer shell may lack cross-links.

Those of ordinary skill in the art would be knowledge of techniques to form a polymer shell on a polymer matrix (e.g., polymer particle). For example, a shell may be formed around the polymer matrix using a coaxial double needle geometry comprising an interior needle portion surrounded by an exterior needle portion. The polymer matrix or precursor may be in the interior needle portion and the shell material may be in the exterior portion, such that a droplet released from the needle comprises the polymer matrix or precursor at least partially surrounded (e.g., completely surrounded) by the shell material. In some such embodiments, the polymer matrix precursor and/or shell material may be crosslinked by exposing the droplet to a crosslinking agent (e.g., divalent ion).

In general, any suitable polymer molecules may be used to form the polymer matrices. In some embodiments, the polymer molecules may be selected based on the intended use of the agent. For instance, in some embodiments, the polymer molecule may be selected based on its compatibility with pharmaceutical applications and other consumer products (e.g., cosmetics, food).

The polymer molecules are generally extended molecular structures comprising backbones which optionally contain pendant side groups, wherein the term backbone is given its ordinary meaning as used in the art, e.g., a linear chain of atoms within the polymer molecule by which other chains may be regarded as being pendant. Typically, but not always, the backbone is the longest chain of atoms within the polymer. A polymer may be a co-polymer, for example, a block, alternating, or random co-polymer. A polymer may also comprise a mixture of polymers. In some embodiments, the polymer may be acyclic or cyclic. A polymer may be cross-linked, for example through covalent bonds, ionic bonds, hydrophobic bonds, and/or metal binding. Polymer molecules may be obtained from natural sources or be created synthetically.

An exemplary, non-limiting list of polymer molecules that are potentially suitable for use in the invention includes polysaccharides (e.g., alginate); polynucleotides; polypeptides; peptide nucleic acids; polyurethane; polyamides; polycarbonates; polyanhydrides; polydioxanone; polyacetylenes and polydiacetylenes; polyphosphazenes; polysiloxanes; polyolefins; polyamines; polyesters; polyethers; poly(ether ketones); poly(alkaline oxides); poly(ethylene terephthalate); poly(methyl methacrylate); polystyrene; poly(lactic acid)/polylactide; poly(glycolic acid); poly(lactic-co-glycolic acid); poly(caprolactone); polysaccharides such as starch; poly(orthoesters); poly(anhydrides); poly(ether esters) such as polydioxanone; poly(carbonates); poly(amino carbonates); and poly(hydroxyalkanoates) such as poly(3-hydroxybutyrate) and poly(3-hydroxybutyrate-co-3-hydroxyvalerate) and derivatives and block, random, radial, linear, or teleblock copolymers, cross-linkable materials such as proteinaceous materials and/or blends of the above. Also suitable are polymer molecules formed from monomeric alkylacrylates, alkylmethacrylates, alpha-methylstyrene, vinyl chloride and other halogen-containing monomers, maleic anhydride, acrylic acid, acrylonitrile, and the like. Monomers can be used alone, or mixtures of different monomers can be used to form homopolymers and copolymers. Other potentially suitable polymer molecules are described in the Polymer Handbook, Fourth Ed. Brandrup, J. Immergut, E. H., Grulke, E. A., Eds., Wiley-Interscience: 2003, which is incorporated herein by reference in its entirety.

The polymer molecules may have any suitable molecular weight. For example, in some embodiments, the polymer molecules may have an average molecular weight greater than 1000 Da, in certain embodiments greater than 5000 Da, in certain embodiments greater than 10000 Da, in certain embodiments greater than 20000 Da, in certain embodiments greater than 50000 Da, in certain embodiments greater than 100000 Da, in certain embodiments greater than 500000 Da, or in certain embodiments greater than 1000000 Da. In some embodiments, the polymer molecules may have at least 5 subunits, in certain embodiments at least 10 subunits, in certain embodiments at least 20 subunits, in certain embodiments at least 30 subunits, in certain embodiments at least 50 subunits, in certain embodiments at least 100 subunits, in certain embodiments at least 500 subunits, in certain embodiments at least 1000 subunits, or in certain embodiments at least 5000 subunits.

In some embodiments, polymer molecules may be biodegradable. In other embodiments, a polymer may be non-degradable. In embodiments where the polymer matrices are to be comprised in a composition for administration to a subject, the polymer molecules may be non-toxic, bioabsorbable, and/or unmodified or modified a naturally occurring polymer molecule (e.g., from a plant, from an animal).

In some cases, the polymer molecule may form a hydrogel. As used herein, the term hydrogel is given its ordinary meaning as used in the art, e.g., a network of polymer chains in an aqueous dispersion medium. In some embodiments, a hydrogel may comprise a plurality of cross-linked polymer molecules. In some cases, a hydrogel polymer matrix is formed by crosslinking the polymer molecules. Non-limiting examples of polymer molecules capable of forming hydrogels include polysaccharides (e.g., alginate), silicon-containing polymers, polyacrylamides, cross-linked polymers (e.g., polyethylene oxide, polyAMPS and polyvinylpyrrolidone), polyvinyl alcohol, acrylate polymers (e.g., sodium polyacrylate), and copolymers with an abundance of hydrophilic groups. In some cases, the polymer molecules may form an organogel, such that the resulting polymer matrices may be swollen by addition of an organic solvent. In some cases, the polymer matrices are a plurality of porous hydrogel particles. In general, the polymer matrix may be formed by cross-linking the polymer molecules. In general, any suitable cross-linking method may be used. For instance, charged polysaccharides (e.g., alginate) may be ionically cross-linked to form a polymer matrix. Those of ordinary skill in the art would be knowledge of suitable cross-linking methods.

In some embodiments, the polymer molecules may form a gel. As used herein, the term gel is given its ordinary meaning in the art and refers to polymer molecules that may be cross-linked to form a network, wherein the network may be able to trap and contain fluids. Depending on the level of crosslinking, various properties of a particular gel can be tailored. For example, a highly cross-linked gel may generally be structurally strong and may resist releasing fluid under pressure. Those of ordinary skill in the art would be able to identify methods for modulating the degree of crosslinking in such gels.

In some embodiments, the polymer molecules may comprise functional groups capable of interacting with another polymer molecule and/or a cross-linking agent. In general, the polymer molecules may have any suitable functional groups.

In general, a wide variety of agents may be crystallized using the methods, described herein. In some embodiments, the agent is a molecular species used in consumer products, such as pharmaceuticals, cosmetics, and/or food products. In some embodiments, the agent is a small molecule (e.g., organic), inorganic salt, a macromolecule, biomolecules (e.g., protein, enzyme), and/or combinations thereof.

The methods, compositions, and/or systems of the present invention may find application relating to pharmaceutical compositions and/or methods, when the agent is a pharmaceutically active agent. The composition may be isolated and used in a variety of application, e.g., use in a pharmaceutical composition for administration to a subject. In addition, a pharmaceutically active agent primarily encapsulated in a particulate polymer carrier, as described herein, may be used directly in a pharmaceutical composition, reducing or eliminating typical processing steps. For example, the particulate carriers may be bound to form a tablet.

The resulting pharmaceutical composition may be provided to a subject. In some cases, prior to administration to the subject, the pharmaceutical composition may be formed into a pharmaceutical product suitable for administration. For example, the particles may be contained in a capsule (e.g., including gel capsules), as a tablet, in a solution (e.g., for injection), etc.

In some embodiments, methods are provided for administering the particulate polymer carriers comprising a pharmaceutically active agent to a subject. In some cases, the method comprises providing crystals of a pharmaceutically active agent primarily encapsulated in the particulate polymer carriers, due to crystallization of the agent in the presence of the carrier; and administering the plurality of particulate polymer carriers to the subject (e.g., a human).

The term “small molecule” is art-recognized and refers to a composition which has a molecular weight of less than about 2000 g/mole, or less than about 1000 g/mole, and even less than about 500 g/mole. Small molecules may include, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides, or polypeptides. In some cases, the small organic molecule is a pharmaceutically active agent (i.e., a drug). A pharmaceutically active agent may be any bioactive agent. In some embodiments, the pharmaceutically active agent may be selected from “Approved Drug Products with Therapeutic Equivalence and Evaluations,” published by the United States Food and Drug Administration (F.D.A.) (the “Orange Book”). In a particular embodiment, the pharmaceutically active agent is aspirin or acetaminophen.

The compositions and/or crystals described herein may be used in “pharmaceutical compositions” or “pharmaceutically acceptable” compositions, which comprise a therapeutically effective amount of one or more of the polymers or particles described herein, formulated together with one or more pharmaceutically acceptable carriers, additives, and/or diluents. The pharmaceutical compositions described herein may be useful for diagnosing, preventing, treating or managing a disease or bodily condition including conditions characterized by oxidative stress or otherwise benefitting from administration of an antioxidant. Non-limiting examples of diseases or conditions characterized by oxidative stress or otherwise benefitting from administration of an antioxidant include cancer, cardiovascular disease, diabetes, arthritis, wound healing, chronic inflammation, and neurodegenerative diseases such as Alzheimer Disease.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid, gel or solid filler, diluent, excipient, or solvent encapsulating material, involved in carrying or transporting the subject compound, e.g., from a device or from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, a “subject” or a “patient” refers to any mammal (e.g., a human), for example, a mammal that may be susceptible to a disease or bodily condition. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition.

Those of ordinary skill in the art can select suitable substantially immiscible fluids, using contact angle measurements or the like, to carry out the techniques of the invention.

A “droplet,” as used herein, is an isolated portion of a first fluid that is completely surrounded by a second fluid. In some cases, the first fluid and the second fluid are substantially immiscible. It is to be noted that a droplet is not necessarily spherical, but may assume other shapes as well, for example, depending on the external environment. The diameter of a droplet, in a non-spherical droplet, is the diameter of a perfect mathematical sphere having the same volume as the non-spherical droplet. The droplets may be created using any suitable technique, as previously discussed.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

The following examples are intended to illustrate certain embodiments of the present invention, but do not exemplify the full scope of the invention.

### EXAMPLES

Nucleation of crystalline materials is omnipresent in nature and industrial practice, specifically, in the chemical and pharmaceutical industry. A promising direction for controlling crystallization is to target nucleation, a critical step in the crystallization process, by designing heteronucleant materials capable of influencing crystallization through selective interactions. However, in industrial practice particularly in pharmaceutical industry, moieties to be crystallized are diverse in chemical structure and accordingly in physical properties such as solubility. The diversity of the moieties along with the demand encountered in industrial practice for biocompatible crystallization promoters introduces additional constraints on the design of biocompatible materials capable of influencing nucleation behavior, crystal formation, crystal size, and morphology. The heteronucleant material designed for industrial practice should be biocompatible, capable of controlling crystallization, capable of carrying industrially relevant amounts of crystalline material (e.g. pharmaceutical in crystalline form), and applicable to hydrophobic and hydrophilic moieties. These examples describe a composite biocompatible hydrogel capable of controlling nucleation from solution, due to rational design of the microstructure and chemical makeup of hydrogel particles, and capable of carrying industrially relevant amounts of water soluble and insoluble pharmaceuticals in chemically distinct environments within composite hydrogel.

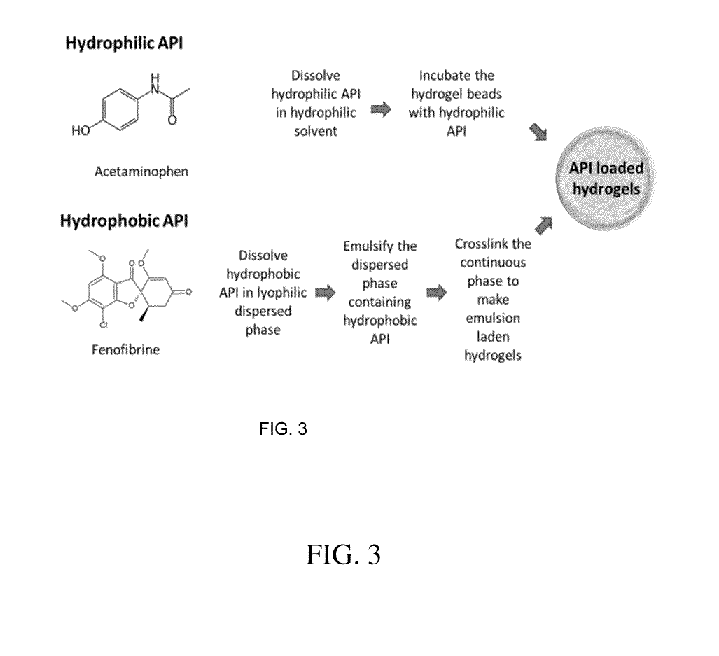

In these examples, methods for designing nanostructure and chemical makeup of biocompatible alginate (ALG) hydrogel particles capable of (i) controlling nucleation from solution and (ii) carrying crystalline active pharmaceutical ingredients (API) of diverse chemical nature (iii) controlling size of the crystals in the final formulation are described. Additionally, the control of nucleation kinetics of the model hydrophilic active pharmaceutical ingredient (Acetaminophen, ACM) from solution and encapsulation of the active pharmaceutical ingredient into the hydrogel through equilibrium partitioning as a function of mesh size is demonstrated. A hydrophobic model active pharmaceutical ingredient (Fenofibrate, FEN) is described that uses, emulsion laden composite hydrogels synthesized for both encapsulation and crystallization. The composite hydrogels were capable of encapsulating and crystalizing the hydrophobic active pharmaceutical ingredient inside emulsion droplets and carrying industrially relevant amounts of active pharmaceutical ingredient. Furthermore, the size of crystals was controlled by adjusting the droplet size and concentration of active pharmaceutical ingredient. FIG. 3 is a schematic illustration of the methods used to form hydrogels loaded with hydrophobic active pharmaceutical ingredients and hydrogels loaded with hydrophilic active pharmaceutical ingredients.

### Example 1

This example describes the synthesis formation of biocompatible hydrogels with controlled microstructure. In an effort to create a pharmaceutically acceptable nucleation step active hydrogel capable of carrying active pharmaceutical ingredients of distinct polarities, a biocompatible polysaccharide used in food, and pharmaceutical industry isolated from brown algae, alginate (ALG) was investigation. Alginate is a linear copolymer, consisted of b-D-mannuronic acid (M) and its C-5 epimer, a-L-guluronic acid (G), arranged in a blockwise pattern. Alginate gel formation can be induced by lowering pH or by adding various divalent cations, in particular Ca⁺², which crosslinks a pair of G blocks within the alginate chains. Despite its utility for carrying hydrophilic drugs, alginate has a hydrophilic nature and accordingly hydrophobic drugs cannot be solubilized or loaded into hydrogel, such alginate, in hydrophilic solvents. To overcome this limitation, composite hydrogels were designed by introducing hydrophobic regions inside hydrogel network by encapsulated nanoemulsion droplets in the hydrogel network. The nanoemulsions provide isolated microenvironments that are chemically different than their surrounding network.

### Example 2

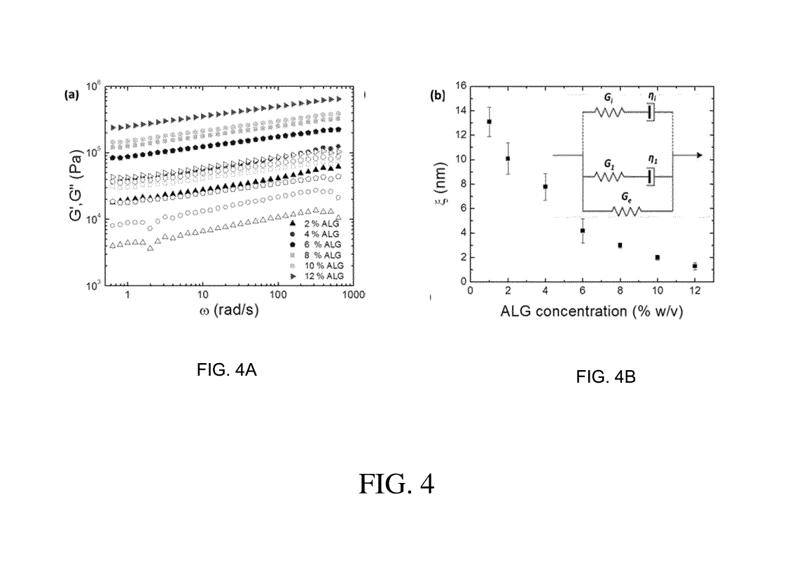

This example describes the characterization of alginate hydrogel formed as described in Example 1. Alginate hydrogels were characterized by evaporation to measure relative solvent content and rheological measurements to estimate the mesh size. For the evaporation measurements, spherical alginate shaped beads (FIG. 4A) were used whereas for rheological measurements disk shaped hydrogels were prepared due to the requirements of the rheometer. Both hydrogel geometries were Ca²⁺ saturated to completely crosslink alginate hydrogels. The evaporation measurements provided information on the weight ratio of solvent to hydrogel bead which was then converted to the volume ratio of hydrogel as the volume of the bead was known a priori. To determine the weight ratio, the hydrogels beads (approximately 20 beads) were first pat dried and weighed and then a microscope image was taken to estimate the size of the hydrogel beads (FIG. 4A). The beads were then placed in a vacuum oven set to 120° C. over night to evaporate the solvent. The weight of the dried hydrogel was measured after evaporation. The difference between the weight of the wet hydrogel and the dry hydrogel was recorded as the weight of evaporated solvent. The weight of the evaporated solvent was converted to volume of evaporated solvent as the density of the solvent was known. The weight and volume ratio of the solvent to the alginate is given in FIG. 4B. All the measurements are performed in triplicates. FIG. 4A shows rheological measurements for alginate hydrogels with different alginate concentrations. FIG. 4B shows the average mesh size based on alginate concentration and a mechanical illustration of Maxwell model for calculating mesh size (inset).

The characterization of mesh size for alginate hydrogels with different alginate concentrations was performed by oscillatory rheology. Disk shaped alginate gels with different alginate concentrations, namely, 2, 4, 6, 8, 10 and 12% (w/v), were prepared in calcium-saturated conditions and their rheological response was recorded (FIG. 4A). In all alginate concentrations considered, the storage (G′) modulus was monotonous and significantly higher than the loss modulus (G″), indicating strong gels behavior. Frequency sweep measurements at a fixed stain (0.05%) were modeled in terms of the generalized Maxwell model. The model utilized was composed of a sequence of elements in parallel (spring and dashpot) to which an additional spring has been added (FIG. 4B). The use of the generalized Maxwell model to describe the alginate system allowed the shear modulus, G and mesh size (ξ) to be determined.

### Example 3

This example describes a method of controlling the nucleation kinetics of hydrophilic active pharmaceutical ingredient by adjusting hydrogel mesh size. No study has been reported on controlling kinetics of nucleation from solution with polymers of tunable nanostructure. Moreover, the effects of pore sizes on the rate of nucleation from solution have not been experimentally studied. In addition, the effect of pore chemistry on nucleation has been largely neglected. Overall, mechanistic understanding of nucleation from solution in nanoconfinement required the design polymers with the proper nanostructure and chemistry to control crystallization is inadequate.

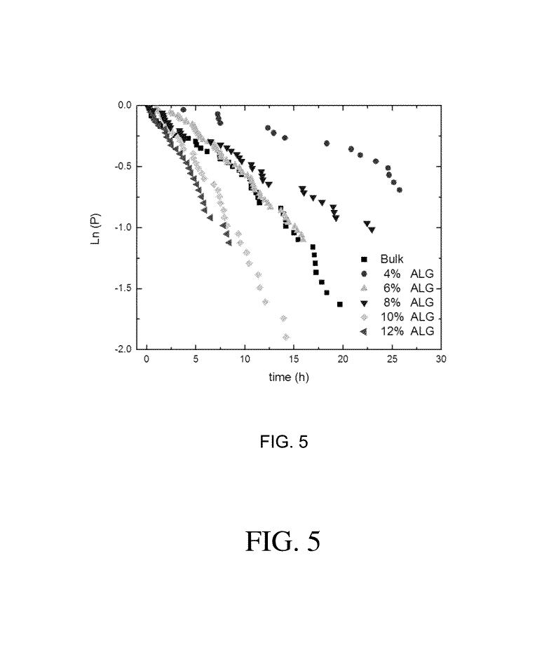

To enable rational design of hydrogels for controlling crystallization of hydrophilic active pharmaceutical ingredient the effect of mesh size on nucleation kinetics was systematically investigated by changing the mesh size via polymer concentration as seen in FIG. 5. In the crystallization solvent, the mesh sizes of these hydrogels ranged from 1 nm to 12 nm. It was found that by changing the mesh size of hydrogel a nucleation event could be enhanced with respect to bulk nucleation. Thus, tuning the microstructure of hydrogels can be used to enhance nucleation from solution.

FIG. 5 shows a graph of time as a function of the natural logarithm of the nucleation induction probability for crystallizations in the presence of alginate particles at different percent composition (4%, 6%, 8%, 10%, and 12%) and the absence of alginate particles versus time.

Nucleation kinetics of model compounds was investigated by measuring the nucleation induction time probability distribution. As used in this example, nucleation induction time is defined as the time elapsed prior to the formation of a detectable amount of the new crystalline phase. Without being bound by theory, it is believed that nucleation induction time is a useful indicator of the surface nucleation activity because nucleation induction time can be dramatically shortened when the presence of an interface lowers the free energy barrier of nucleation. A large number of experiments were performed to obtain the probability distribution of nucleation induction time. To obtain the average induction time τ, statistical analysis on the induction time data was conducted based on the understanding that nucleation follows a Poisson distribution. According to Poisson statistics, the probability for a nucleation event to occur beyond time t is P=exp(−t/τ), which implies that the fraction of vials without nucleation at time t exponentially decays as a function of time, where the scaling factor for time is the average induction time. The induction times are given in Table 1.

TABLE 1
Summary of average induction time (τ) measurements for alginate
particles of different concentrations used as heteronucleants during
crystallization of ACM from a supersaturated ethanol solution at 10° C.

| sample | τ (hrs.) | error (hrs.) | linearity |
|---|---|---|---|
| control | 15.3 | 0.31 | 0.99 |
| 4% alginate | 24.7 | 1.4 | 0.92 |
| 6% alginate | 12.8 | 0.23 | 0.97 |
| 8% alginate | 16.5 | 0.50 | 0.95 |
| 10% alginate | 9.5 | 0.18 | 0.98 |
| 12% alginate | 7.7 | 0.10 | 0.99 |

### Example 4

Example 4 describes a method of controlling the loading of hydrophilic active pharmaceutical ingredient via mesh size of hydrogel.

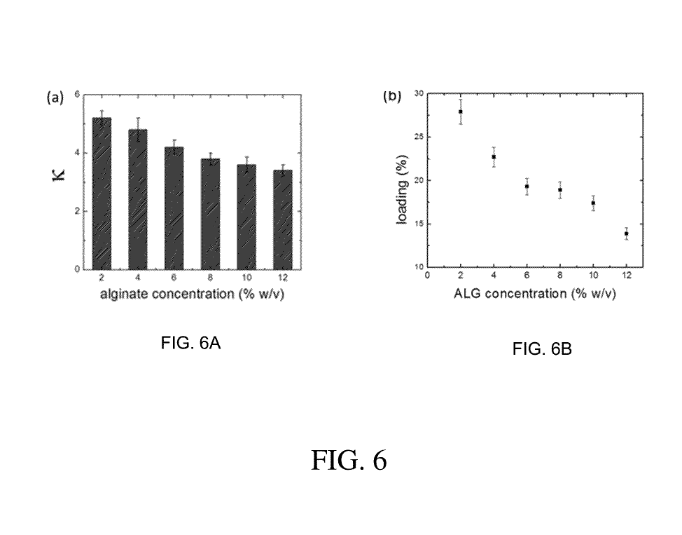

It has been demonstrated that hydrophilic active pharmaceutical ingredient partitioning into alginate hydrogel can be controlled by polymer concentration (% alginate by weight prior to crosslinking) i.e., mesh size of hydrogel (FIG. 6A and FIG. 6B show the ACM loading in hydrated hydrogels calculated by equilibrium partitioning for various alginate concentrations and the percent loading of active pharmaceutical ingredient in alginate hydrogel in weight percent). The hydrogels immersed in a polar solvent in equilibrium with solid active pharmaceutical ingredient could load up to 27 wt. % of hydrophilic active pharmaceutical ingredient through equilibrium partitioning. To determine the amount of drug loaded a known amount of the particles was transferred to a known amount of solvent (water) and stirred at a constant temperature. The dissolution or release of the drug was monitored over time using UV-vis spectrometry and the concentration of the drug at equilibrium was determined using its absorbance. It was observed that the amount of drug loaded at lower alginate concentration was more than twice the amount loaded at higher alginate concentrations (FIG. 6). Without being bound by theory, it is believed that this effect might be due to the larger pore size and size distribution of the lower alginate concentrations and also the solute partition coefficient which decreases with increasing alginate concentration.

### Example 5

This example describes the formulation of composite hydrogels for crystalizing hydrophobic active pharmaceutical ingredient.

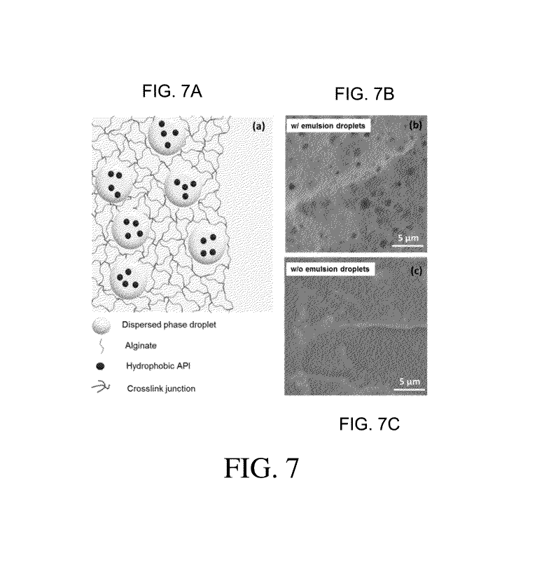

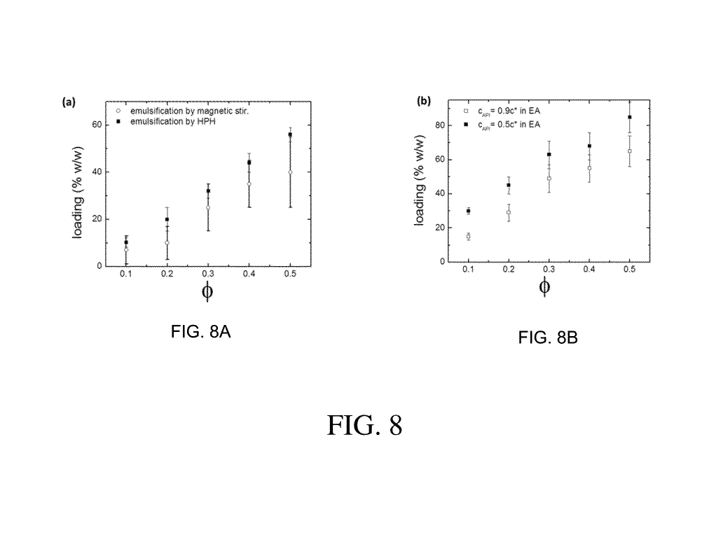

Composite hydrogels containing emulsion droplets were embedded in hydrogel matrix, effectively creating hydrophobic regions in an otherwise hydrophilic alginate network. The dispersed phase was emulsified in nano and micron size droplets using heptane and ethyl acetate as a solvents in order to load the drugs inside the alginate particles (FIG. 7A shows an illustration describing emulsion laden composite hydrogels loaded with hydrophobic active pharmaceutical ingredient, fenofibrate (FEN). Environmental scanning electron microscope images confirmed the presence of the emulsified droplets inside the alginate particle (FIG. 7B-C show cross-section of an environmental scanning electron microscopy (eSEM) images of alginate hydrogels (B) with emulsion droplets and (C) without emulsion droplets serving as control for panel (B). The continuous phase contained 2% alginate and the volume fraction of the dispersed phase was 30% (v/v) FEN in heptane). The hydrophobic compound utilized as a model active pharmaceutical ingredient for the hydrophobic drugs was fenofibrate (FEN). The emulsified solution was added to alginate solutions having various concentrations. The emulsified solution containing the fenofibrate and alginate solution was cross-linked in situ in a calcium chlorine solution. The emulsion laden composite hydrogels were formed with different emulsion volume fractions ranging between 10% to 50% (FIG. 8). The emulsion laden composite hydrogels had the ability to crystalize FEN inside droplets and also demonstrated tunable loading up to 85% by weight active pharmaceutical ingredient in dried hydrogel.

### Example 6

This example describes the loading characteristics of composite hydrogel with respect to hydrophobic active pharmaceutical ingredients.

The utility of loading industrially relevant amounts of hydrophobic drug and the ability to control the amount of drug loaded by varying volume fraction of dispersed phase, dispersed phase chemical makeup, and concentration of active pharmaceutical ingredient in dispersed phase was demonstrated. First the advantage of using high pressure homogenization (HPH) for emulsification step (FIG. 8 was demonstrated). In FIG. 8A, it is shown that with HPH, a smaller variation in loading was achieved compared to emulsification with magnetic stirring. This was attributed to smaller variations in droplet size obtained in HPH than magnetic stirring, therefore it was concluded that HPH was a better suited for pharmaceutical production where dosage consistency is of utmost importance.

Secondly, the ability to control amount of hydrophobic drug loaded in hydrogel on dry basis by changing volume fraction and concentration of active pharmaceutical ingredient in dispersed phase (Ethyl Acetate (EA)) was demonstrated. For each loading measurement of a given volume fraction (φ), two batches of emulsion laden hydrogels were prepared with the same method of emulsification followed by crosslinking (i.e., a reference batch without active pharmaceutical ingredient and a test batch with active pharmaceutical ingredient). Both batches contained approximately 200 mg of alginate beads and were pat-dried and weighted. Both batches were placed in vacuum oven and dried over two days at 140° C. which is above the boiling point of dispersed phase (boiling point of heptane 98° C., ethyl acetate 77.1° C. at 1 bar). Initially heptane was used as the continuous phase; later in an effort to maximize the loading ethyl acetate was used as the continuous phase. The solubility of FEN in ethyl acetate was considerably larger than in heptane (9 mg/mL and 600 mg/mL). Loading was defined as the difference in weight between the hydrogels containing the active pharmaceutical ingredient carrying emulsions and reference batch the without the active pharmaceutical ingredient divided by weight of active pharmaceutical ingredient carrying emulsion laden hydrogel on dry solute basis as:

$$Loading \% = \frac{{W}_{API carrying emulsion laden hydrogel} - {W}_{reference emulsion laden hydrogel}}{{W}_{API carrying emulsion laden hydrogel}} * 100.$$

To estimate the variation in loading and significance of variation in ten replicates were performed.

FIG. 8A is a graph showing the loading of FEN in heptane emulsion laden hydrogels measured by evaporation method. FIG. 8B is a graph showing the loading of FEN in ethyl acetate with two different FEN concentrations for varying emulsion volume fractions measured by evaporation method.

### Example 7

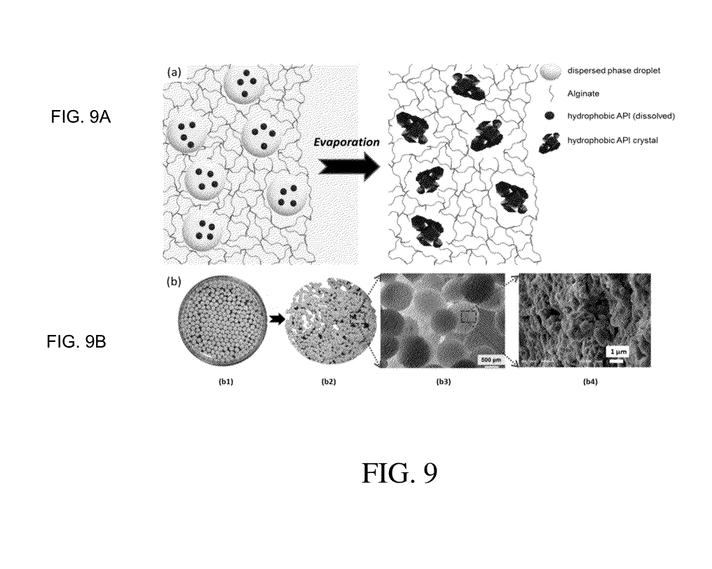

This example describes the control of crystal size using composite hydrogels. The distribution in crystal size of pharmaceuticals in a formulation is critical to the pharmaceuticals dissolution rate and accordingly pharmokinetic performance in body. Smaller crystals provide a larger surface area for a given active pharmaceutical ingredient mass. The larger area promotes quicker dissolution allowing active pharmaceutical ingredient to dissolve more quickly in vivo. For hydrophobic drugs with low aqueous solubility, there are limited methods for achieving a reduced crystal size. The state of the art methods such as mechanical milling or spray freezing into liquid are harsh treatments that can induce formation of metastable polymorphs. In this example, the development of composite hydrogel for controlling crystal size is described and the demonstrated control of the dissolution rate for an hydrophobic active pharmaceutical ingredient is demonstrated. The composite hydrogels consisted of trapped emulsion droplets carrying active pharmaceutical ingredient in a biocompatible polymer (alginate) matrix. By controlled evaporation of continuous phase and dispersed phase, crystallization of active pharmaceutical ingredient with in hydrogel matrix was induced (FIG. 9). Control of emulsion droplet size and active pharmaceutical ingredient concentration within the emulsion droplet allowed crystals as small as 300 nm and as large as 0.5 mm to be produced.

FIG. 9 shows a pictorial demonstration of producing embedded crystals in dried hydrogel matrix through controlled evaporation of composite hydrogel where the crystal size was controlled by droplet volume and concentration of active pharmaceutical ingredient. First hydrophobic active pharmaceutical ingredient (FEN) was dissolved in an organic phase (FDA Class III solvent Anisole). Then the organic phase carrying the hydrophobic active pharmaceutical ingredient was dispersed in an aqueous continuous phase with a suitable surfactant (Pluronic® F-68). Due to preferential partitioning of the hydrophobic active pharmaceutical ingredient to the dispersed organic phase, the active pharmaceutical ingredient was predominantly in the organic phase; only a minute fraction of the active pharmaceutical ingredient partitioned into the continuous aqueous phase. The Na-alginate dissolved in continuous phase was then ionically crosslinked with Ca²⁺ ions in order to trap dispersed phase droplets containing the active pharmaceutical ingredient in the organic phase in the hydrogel matrix. The trapped dispersed phase inside the hydrogels matrix formed composite hydrogels depicted in FIG. 9A.

Through controlled evaporation at 60° C., evaporation of both the dispersed phase and continuous phase was induced. Once all liquids were evaporated the crystals were entrapped in dried hydrogel matrix. The crystals were larger than the mesh size of the polymer matrix, but could not grow above the size of emulsion due to confinement. FIG. 9B shows the composite hydrogels in hydrated form (b1), after drying (b2) optical microscope image of dried composite beads (b3), and a high magnification SEM image of dried composite hydrogel sliced cross-section (b4).

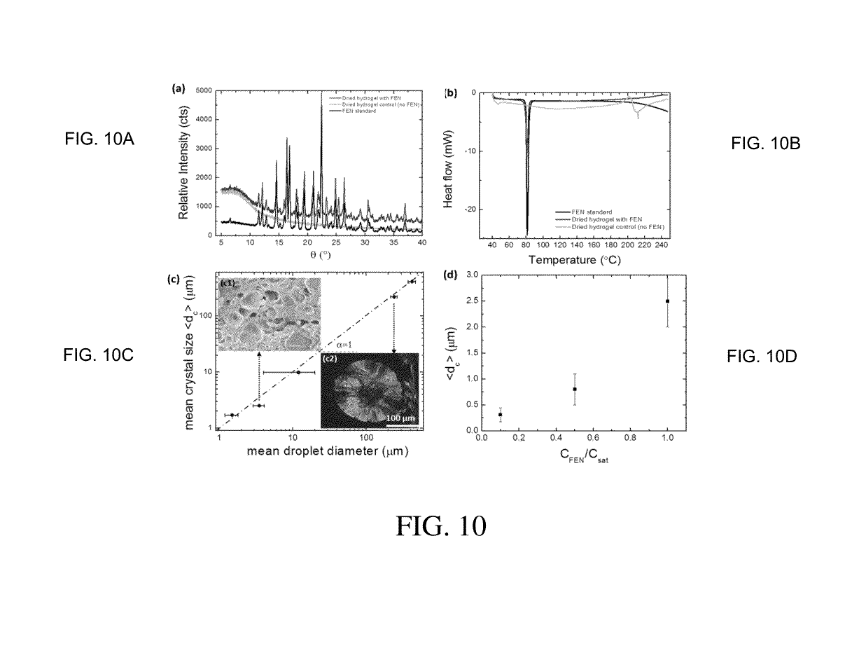

To investigate if the active pharmaceutical ingredient was crystalline inside the dried composite hydrogel, powder X-ray diffraction scattering (PXRD) and differential scanning calorimetry (DSC) was used. FIG. 10A shows PXRD pattern from the FEN standard, dried composite hydrogel carrying FEN and control dried composite hydrogel without FEN. The XRD patterns of standard and dried composite hydrogel carrying FEN indicated the crystalline structure of FEN inside dried hydrogels. DSC also showed FEN standard, dried composite hydrogel carrying FEN and control dried composite hydrogel without FEN. The melting point of FEN standard and dried composite hydrogel carrying FEN coincided at 81° C. (FIG. 10B). These findings together with PXRD results proved that FEN was crystalline inside dried composite hydrogels.

The crystal size was controlled by the droplet size and the concentration of the active pharmaceutical ingredient in dispersed phase. When the dispersed phase was saturated with the active pharmaceutical ingredient (i.e., concentration of FEN in dispersed phase is equal to saturation concentration of FEN in the dispersed organic phase (C_{FEN}/Cₛₐₜ=1)), the droplet size of the dispersed phase dictated the size of the crystals. Using three different emulsification techniques, namely high pressure homogenization, bulk emulsification and millifluidics, emulsions ranging between 1.5 micron to 0.5 mm were prepared. As seen in FIG. 10C, the mean droplet size and mean crystal size were equal within the error bounds for three different emulsification techniques. In addition to droplet size, the crystal size was controlled by controlling the concentration of the active pharmaceutical ingredient within the dispersed phase. By decreasing the concentration of C_{FEN} in the dispersed phase, the crystal size was decreased below the droplet size. Hence controlling C_{FEN}/Cₛₐₜ, allowed control over the crystal size and allowed crystals smaller than size dictated by the droplet size to be formed.

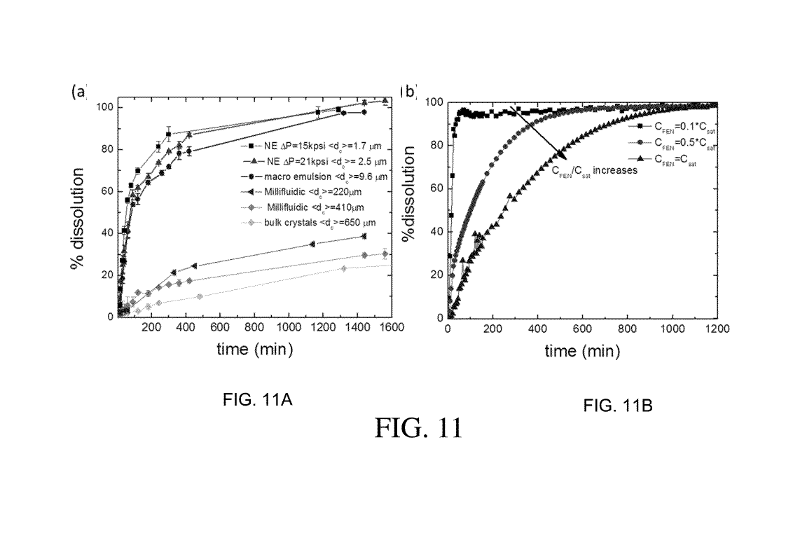

By controlling the crystal size, the dissolution profiles of dried composite hydrogels could also be controlled. Dried composite hydrogels with different crystal sizes exhibited different dissolution profiles. Smaller crystal sizes give larger surface area per unit mass and this led to faster dissolution. FIG. 11A shows dissolution profiles of dried hydrogels with different mean crystal sizes (C_{FEN}/Cₛₐₜ=1). FIG. 11B shows that the dissolution profile could be tuned by controlling concentration of active pharmaceutical ingredient inside emulsion droplets and hence the crystal size. Composite hydrogels that had the same entrapped dispersed phase droplet size but different FEN concentrations resulted in different dissolution rates.

FIG. 11A shows the dissolution profile for different crystal sizes where organic phase was saturated by FEN i.e. C_{FEN}=C_{FEN}^{SAT}. FIG. 11B shows the dissolution profile where the crystal size was controlled by concentration of FEN in emulsion droplets of given sizes.

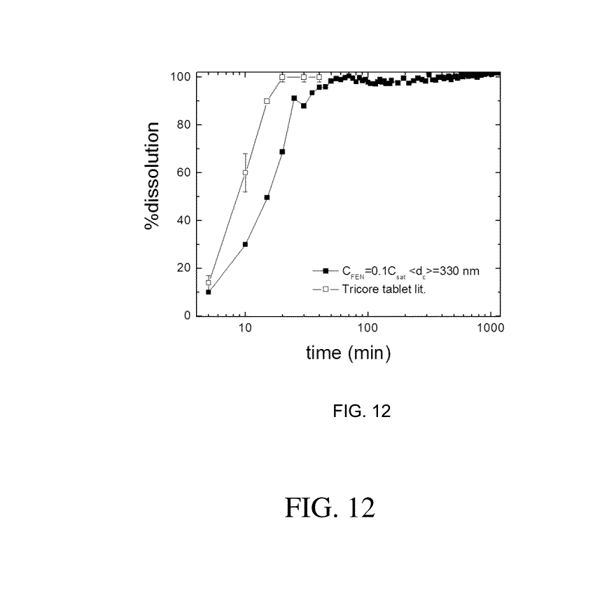

In some embodiments, a fast dissolution time is important. The results above were compared to commercially available formulation TriCor tablets. FIG. 12 shows the semilog plot dissolution profiles of FIG. 11B compared to literature. FIG. 12 shows that the dissolution rates were comparable to commercially available formulations.

The state of the art in industrial practice to control nucleation involves methods such as adjusting supersaturation levels, temperature profiles, crystallization solvent, stirring speed, seeding with existing active pharmaceutical ingredient crystals, etc. However, the nucleation behavior remains largely unpredictable due to the presence of unregulated foreign surfaces that lower nucleation energy barrier. The Examples describe a biocompatible hydrogel excipient particles with morphology designed specifically to directly control the nucleation kinetics and crystal outcome for hydrophobic and hydrophilic active pharmaceutical ingredient. The methodology developed was amenable to continuous manufacturing, particularly composite hydrogels provided compartmentalized units where crystallization could be achieved. These compartmentalized units were accessible as they were embedded in a hydrogel. Hence crystallization could be induced either by temperature shock, evaporation, or chemical interference.

The current state of the art in improving dissolution rates of hydrophobic active pharmaceutical ingredient are based on decreasing crystal size of mm size native active pharmaceutical ingredient crystals with harsh milling or spray drying methods. Due to their harsh nature with considerable energy input, these methods are known to introduce polymorphism i.e. formation of metastable polymorphs. The Examples describe a methodology of using composite hydrogels in mild methods where the active pharmaceutical ingredient crystals were formed inside a hydrogel matrix hence metastable polymorphs were avoided. It was found that the crystal size, which dictates the dissolution profile, could be controlled by controlling droplet size of trapped dispersed phase and concentration of active pharmaceutical ingredient. Relatively high loading of submicron crystals at (up to 20% active pharmaceutical ingredient on dry basis) was also described.

Furthermore, the polymer matrix in which crystals were embedded provided natural protection against mechanical effect that could disturb crystal morphology. Once the crystals were formed they were protected from mechanical effects, because the crystals were surrounded by the hydrogel matrices. Additional protective layers could be added by using coaxial needles with ease.

The ability of the developed biocompatible hydrogel excipients to carry pharmaceutically relevant amounts of both hydrophobic and hydrophilic active pharmaceutical ingredient was unique. It was demonstrated that the developed alginate hydrogels and composite alginate hydrogels containing nanoemulsion droplets could carry active pharmaceutical ingredient using two separate mechanisms. Alginate hydrogels' ability to absorb and concentrate hydrophilic active pharmaceutical ingredient due to equilibrium partitioning within the interior of the particles was unique. Such partitioning is not readily obtained with any other type of previously used excipient (SAMs, crystalline surfaces, glass, solid polymers, etc.). Conversely, the composite hydrogels containing nanoemulsion droplets were embedded in the hydrogel matrix trapped large amounts of hydrophobic active pharmaceutical ingredient due to lyophilicity of nanoemulsion. Furthermore, aforementioned mechanisms could be utilized to orthogonally load composite hydrogels with industrially relevant (up to 85% by dry weight) amounts of hydrophobic and hydrophilic active pharmaceutical ingredient. Also, nanoemulsions carrying different hydrophobic pharmaceuticals could be loaded inside composite hydrogels. The ability to control crystallization and load large amounts of active pharmaceutical ingredient with diverse solubility, individually or orthogonally, make the polymer matrices, described herein, unique.

### Example 8

This example describes applications in which the methods, compositions, and systems described in Examples 1-7 may be used and are prophetic.

The method described in this invention could be applied to designing biocompatible particles to regulate nucleation kinetics, to load active pharmaceutical ingredient of diverse chemical and physical properties and to crystalize hydrophobic active pharmaceutical ingredient with controlled size (appx. 300 nm to 0.5 mm) for pharmaceutical industry, food industry and other industries that require crystallization and delivery of small organic compounds.

Applications in Pharmaceutical Manufacturing of Hydrophilic Active Pharmaceutical Ingredient

Crystallization is extensively used to purify the active pharmaceutical ingredients in the pharmaceutical manufacturing process. After the crystallization step, the active pharmaceutical ingredient crystals are then granulated and blended with excipients before packaging into the final dosage form. Granulation and blending processes are problematic due to their harsh nature where active pharmaceutical ingredient crystals can be broken or aggregated even transformed into a metastable polymorph. Pharmokinetic performance of pharmaceutical is very sensitive to the shape and size of the drug crystals. Hence manufacturing processes where the crystal size and morphology can be controlled and protected from environmental factors is desired.

The methods, compositions, and systems described in Examples 1-7 allowed heterogeneous crystallization of hydrophilic active pharmaceutical ingredient from solution on the surface of an amorphous excipient, so that the resulting active pharmaceutical ingredient-excipient composite particles could either serve as the dosage form themselves, or were agglomerated and formed directly into the final dosage form, thereby eliminating the subsequent granulation, blending, and compaction steps. Furthermore, design of the excipient surface properties to control the active pharmaceutical ingredient nucleation kinetics and final crystal form allowed for better control over the quality and uniformity of the final drug and dosage form.

Application in Pharmaceutical Manufacturing of Hydrophobic Active Pharmaceutical Ingredient

Since 1980, 43% of all newly developed pharmaceuticals are estimated to be extremely insoluble in water. Hence new methodologies for producing hydrophobic active pharmaceutical ingredient are essential in pharmaceutical industry. The current state of art in production of hydrophobic pharmaceuticals is based on granular particle processing that is by definition a batch process or using organic solvents that needs to be removed prior to final packaging.

The composite hydrogels described in the Examples, namely composite hydrogels containing nanoemulsion droplets, were capable of carrying and crystalizing hydrophobic active pharmaceutical ingredient in aqueous environment. Hence the composite hydrogels either served as the dosage form themselves, or were dried into the final dosage form, eliminating the batch granular particle processing. The nanoemulsion droplets acted as hydrophobic regions favoring hydrophobic active pharmaceutical ingredient within hydrophilic hydrogel. This favorable interaction was responsible for pharmaceutically relevant amount (up to 85% by weight active pharmaceutical ingredient in hydrogel on dry basis) of agent that was loaded.

With controlled mild evaporation, composite hydrogels carrying active pharmaceutical ingredient were transformed into final dosage form with controlled active pharmaceutical ingredient crystal size embedded in dried hydrogel(polymer) matrix. The crystal size were controlled by adjusting droplet size and active pharmaceutical ingredient concentration.

Applications in Hydrophobic Active Pharmaceutical Ingredient Delivery

The current state of the art in pharmaceutical industry for improving dissolution rates of notoriously difficult to dissolve hydrophobic active pharmaceutical ingredient involves reducing the size via nanomilling methods or physical absorption into nanoporous matrix in amorphous state. However, these methods are not ideal because the drugs are prone to phase transformation into metastable polymorphs under mechanical stress or to recrystallize since the amorphous form is metastable. Methods, compositions, and/or systems, described herein, demonstrated direct crystallization of hydrophobic active pharmaceutical ingredient in the drug carrier to a desired crystal size, and accordingly, desired dissolution rate.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a polymer particle comprising crystals of a pharmaceutically active agent, wherein the crystals have an average diameter that is greater than an average mesh size of the polymer particle and wherein the average diameter has a coefficient of variation less than or equal to about 10%.

2. The composition of any preceding claim, wherein a polymer particle is cross-linked.

3. The composition of any preceding claim, wherein a diameter of the polymer particle is less than or equal to about 600 microns.

4. The composition of any preceding claim, wherein the polymer particle comprises a naturally occurring polymer.

5. The composition of any preceding claim, further comprising a polymer shell.

6. The composition of any preceding claim, wherein the polymer shell at least partially surrounds the polymer particle.

7. The composition of any preceding claim, wherein the average diameter of the crystals is less than or equal to about 1 micron.

8. The composition of any preceding claim, wherein the coefficient of variation is less than or equal to about 10%

9. The composition of any preceding claim, wherein the average mesh size is than or equal to about 10 nm.

10. The composition of any preceding claim, wherein the ratio of the average mesh size to the average diameter of the crystals is less than or equal to about 0.8:1.

11. The composition of any preceding claim, wherein the ratio of the average mesh size to the average diameter of the crystals is less than or equal to about 0.2:1.

12. The composition of any preceding claim, wherein the pharmaceutically active agent is a small organic molecule.

13. The composition of any preceding claim, wherein a weight percentage of the crystals in the polymer particle is greater than or equal to about 10%.

14. The composition of any preceding claim, wherein a weight percentage of the crystals in the polymer particle is greater than or equal to about 40%.

15. The composition of any preceding claim, wherein the polymer particle is formed from a polysaccharide.

16. The composition of any preceding claim, wherein the polymer particle is a hydrogel.

17. A method, comprising administering to a subject the composition of claim 1.

18. A composition, comprising:
crystals of a pharmaceutically active agent dispersed throughout a cross-linked polymer matrix, wherein the solubility of a polymer matrix precursor in a solvent prior to crosslinking is at least 2 times greater than the solubility of the pharmaceutically active agent in the solvent.

19. The composition of any preceding claim, wherein the solubility of a polymer matrix precursor in a solvent prior to crosslinking is at least 10 times greater than the solubility of the pharmaceutically active agent in the solvent.

20. The composition of any preceding claim, wherein the solubility of a polymer matrix precursor in a solvent prior to crosslinking is at least 100 times greater than the solubility of the pharmaceutically active agent in the solvent.

21. The composition of any preceding claim, wherein the solvent is water.

22. The composition of any preceding claim, wherein the polymer matrix comprises a naturally occurring polymer.

23. The composition of any preceding claim, further comprising a polymer shell, at least partially surrounds the polymer matrix.

24. The composition of any preceding claim, wherein the average diameter of the crystals is less than or equal to about 1 micron.

25. The composition of any preceding claim, wherein the coefficient of variation in the diameter of the crystals is less than or equal to about 10%

26. The composition of any preceding claim, wherein the average mesh size of the polymer matrix is than or equal to about 10 nm.

27. The composition of any preceding claim, wherein the ratio of the average mesh size to the average diameter of the crystals is less than or equal to about 0.8:1.

28. The composition of any preceding claim, wherein the ratio of the average mesh size to the average diameter of the crystals is less than or equal to about 0.2:1.

29. The composition of any preceding claim, wherein the pharmaceutically active agent is a small organic molecule.

30. The composition of any preceding claim, wherein the weight percentage of the crystals in the polymer matrix is greater than or equal to about 20%.

31. The composition of any preceding claim, wherein the weight percentage of the crystals in the polymer particle is greater than or equal to about 50%.

32. The composition of any preceding claim, wherein the polymer matrix comprises a polysaccharide.

33. The composition of any preceding claim, wherein the polymer matrix is a hydrogel.

34. A method, comprising administering to a subject the composition of claim 20.

35. A pharmaceutical composition comprising:
a particulate polymer carrier; and
a pharmaceutically active agent primarily encapsulated by the particulate polymer carrier,
wherein the active agent has been crystallized in the presence of the particulate polymer carrier.

36. A method, comprising administering to a subject the composition of claim 35.

37. The composition of any preceding claim, wherein the diameter of the particulate polymer carrier is less than or equal to about 10 microns.

38. The composition of any preceding claim, wherein the particulate polymer carrier is formed from a naturally occurring polymer.

39. The composition of any preceding claim, comprising a polymer shell.

40. The composition of any preceding claim, wherein the polymer shell at least partially surrounds the particulate polymer carrier.

41. The composition of any preceding claim, wherein the ratio of the average mesh size to the average diameter of the crystals is less than or equal to about 0.8:1.

42. The composition of any preceding claim, wherein the ratio of the average mesh size to the average diameter of the crystals is less than or equal to about 0.2:1.

43. The composition of any preceding claim, wherein the pharmaceutically active agent is a small organic molecule.

44. The composition of any preceding claim, wherein the polymer particle is formed from a polysaccharide.

45. The composition of any preceding claim, wherein the polymer particle is formed from a hydrogel.

46. A method, comprising:
crystallizing a pharmaceutically active agent in a fluid droplet within a polymer particle

47. The method of any preceding claim, wherein the fluid droplet comprises an apolar solvent.

48. The method of any preceding claim, wherein the polymer particle is cross-linked.

49. The method of any preceding claim, wherein the polymer particle has a mesh size less than or equal to about 10 nm.

50. The method of any preceding claim, comprising forming an emulsion.

51. The method of any preceding claim, wherein the polymer particle is in a continuous phase of the emulsion.

52. The method of any preceding claim, wherein the fluid droplet is in a dispersed phase of the emulsion.
